(19) United States
(12) Patent Application Publication
(10) Pub. No.: US 2023/0272323 A1
(43) Pub. Date: Aug. 31, 2023

(54) CELL CULTURING VESSEL AND RELATED METHODS

(71) Applicant: UNIVERCELLS TECHNOLOGIES S.A., Nivelles (BE)

(72) Inventors: Sebastien Rodriguez, Nivelles (BE); Jean-Christophe Drugmand, Wavre (BE)

(21) Appl. No.: 18/028,276

(22) PCT Filed: Sep. 27, 2021
(86) PCT No.: PCT/EP2021/076451

§ 371 Date: Mar. 24, 2023

Related U.S. Application Data
(60) Provisional application No. 63/083,491, filed on Sep. 25, 2020.

(51) Int. Cl. C12M 1/00; C12M 3/00; C12M 1/06; C12M 1/12

(52) U.S. Cl. CPC C12M 23/34; C12M 23/46; C12M 25/18; C12M 27/02

(57) ABSTRACT

A cell culturing container including a fixed bed is provided for forming a bioreactor (100). In one version, a container in the form of a tubular body is formed by first and second interlocking portions (115a, 115b). The container forms a compartment for receiving the fixed bed (122) for culturing cells, which container may be removably inserted into an inner compartment of the bioreactor so as form an outer chamber for circulating fluid to or from the fixed bed. The container may also include a removable base (130) for receiving an agitator (118) for agitating and thus circulating the fluid. Related methods are also disclosed.

This application claims the benefit of U.S. Provisional Pat. Application Ser. No. 63/083,491, filed Sep. 25, 2020, the disclosure of which is incorporated herein by reference.

### TECHNICAL FIELD

This document relates generally to the cell culturing arts and, more particularly, to cell culturing vessels, such as bioreactors.

### BACKGROUND

Some high cell density cell culture vessels, such as bioreactors, employ a fixed bed for the growth of cells that are entrapped therein or thereon. A desirability exists to be able to manufacture such vessels in a modular fashion, such as to allow for ready assembly for use in the field and to provide a high degree of dimensional flexibility (such as variable dimension including height). However, past proposals suffer from undue complexity, as well as a lack of flexibility in the ability to vary the construction of the vessel forming the bioreactor.

Accordingly, a need is identified for an improved modular cell culturing vessel that overcomes the foregoing limitations as well as others.

### SUMMARY

According to one aspect of the disclosure, an apparatus for culturing cells is provided. The apparatus comprises a housing having an inner compartment. A container is provided for removable positioning in the inner compartment of the housing. The container comprises first and second portions adapted to interlock for containing a fixed bed for culturing cells, the container forming a chamber within the inner compartment for circulating fluid to or from the fixed bed.

The first and second portions comprise interlocking male and female couplers. The first and second portions may further include a sealing arrangement forming a tortuous path for fluid flow.

The removable container may include one or more internal grooves for engaging retainers for retaining the fixed bed therein. The container may comprise a central tube, and the retainers adapted for engaging the central tube. Specifically, each of the retainers may include a barb adapted for insertion into an open end of the central tube.

The removable container may further include a third portion for receiving an agitator. The third portion may include a door for receiving the agitator, as well as one or more openings for allowing fluid flow to enter the container. The third portion may be adapted to relasably connect to the container. A retainer comprising a plate with peripheral notches may be provided for allowing fluid to pass to the third portion of the container.

A cap may be provided for engaging the container, the cap comprising a hinged plate. The hinged plate may comprise a releasable locking arrangement for locking with the cap in a closed position.

In any of the foregoing embodiments, the first and second portions comprise halves of the container.

According to a further aspect of the disclosure, an apparatus for culturing cells is provided. The apparatus comprises a fixed bed comprising a retainer at one end and a tube passing centrally through the fixed bed, the tube including an open end for receiving a portion of the retainer. The fixed bed may comprise a spiral fixed bed, and may further include a tubular body for receiving the fixed bed, the tubular body having an internal groove for receiving the retainer. A cap may be provided for engaging the tubular body, the cap comprising a hinged plate, which may further include a releasable locking arrangement for locking with the cap in a closed position.

Still a further aspect of the disclosure relates to a method of assembling a bioreactor. The method comprises interconnecting first and second portions to form a container including a fixed bed, and inserting the container into an inner compartment of a housing to form a chamber therein circulting fluid to or from the fixed bed. The method may further include the step of providing a retainer on opposed ends of the fixed bed prior to the inserting step. Still further, the method may include the step of connecting the retainers to the first and second portions of the container prior to the inserting step, such as by inserting a portion of the retainers into a tube positioned within the fixed bed. The method may further include the step of connecting a base portion including an agitator to the container prior to the inserting step.

### BRIEF DESCRIPTION OF THE DRAWING FIGURES

FIG. 1 is a perspective view of a bioreactor including a cell culturing container according to one aspect of the disclosure;

FIG. 1A is a cross-sectional view of the cell culturing container;

FIG. 2 is a side view of the fixed bed;

FIG. 2A is a top view of the fixed bed of FIG. 2;

FIG. 2B is a partially cutaway view of a portion of the fixed bed of FIG. 2;

FIG. 2C is another partially cutaway view of a portion of the fixed bed of FIG. 2;

FIG. 3 is an exploded view of the portion of the cell culturing container of FIG. 1A;

FIG. 4 is a front perspective view of the cell culturing container of FIG. 1A;

FIGS. 4A, 4B, 4C, 4D, 4E, and 4F are enlarged partial views of upper portions of the cell culturing container of FIG. 1A;

FIGS. 5, 5A, 5B, and 5C are enlarged partial views of a lower portion of the cell culturing of FIG. 1A;

FIGS. 6A, 6B, and 6C are views of portions of the cell culturing container of FIG. 1A;

FIGS. 7A, 7B, and 7C are views of portions of the modular cell culturing container of FIG. 1A;

FIGS. 8A and 8B illustrate one version of a hinged lid or cover for use with a cell culturing container;

FIG. 9 illustrates another version of a hinged lid or cover for use with a cell culturing container;

FIG. 10 illustrates another embodiment of a cell culturing container including an external door;

FIGS. 11 and 12 illustrate additional versions of cell culturing containers;

FIGS. 13 and 14 illustrate a base portion of a cell culturing container; and

FIGS. 15 and 16 illustrate yet another base portion of a cell culturing container.

### DETAILED DESCRIPTION

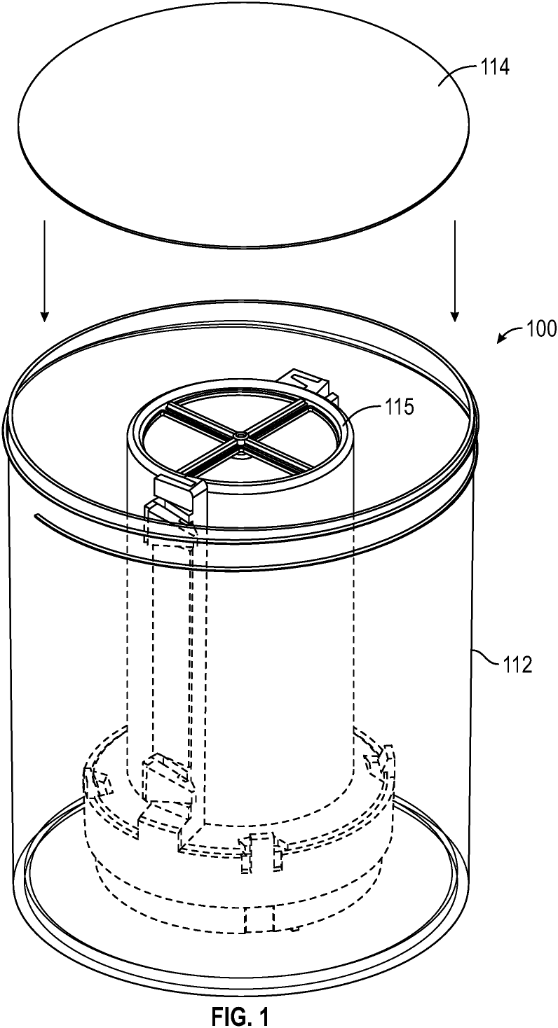

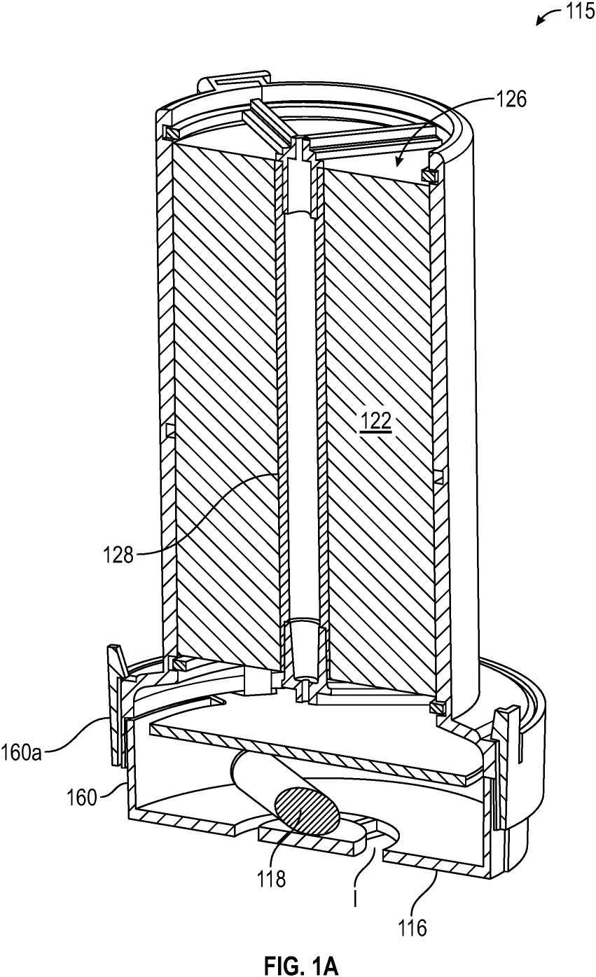

Reference is now made to FIG. 1, which illustrates one embodiment of a fixed bed bioreactor 100 for culturing cells according to one aspect of the disclosure. The bioreactor 100 includes an external casing or housing 112 forming or including an interior compartment for holding a fluid. A cover 114 may also be placed on top of the housing 112 to cover or seal the interior compartment, and may include various openings or ports with removable closures or caps (not shown) for the selective introduction or removal of material, fluid, gas, probes, sensors, samplers, or the like.

Within the interior compartment of the bioreactor housing 112, an inner container 115 may be provided to contain and, therefore, divide the fixed bed-containing portion of the bioreactor from the remaining portion (e.g., outer chamber) devoid of a fixed bed. This container 115, when situated within the housing 112 thus forms an inner compartment or chamber for transmitting a flow of fluid, gas, or both, throughout the bioreactor 100 and, in particular, through the container 115.

As indicated in FIG. 1, the container 115 may include a chamber 116 at or near a base of the bioreactor 100. The chamber 116 may include an agitator for causing fluid flow within the bioreactor 100, such as for example a drop-in, rotatable, non-contact magnetic stir bar 118 or impeller, which in either case forms a centrifugal pump in the bioreactor 100. While not shown, the agitator could also be in the form of an agitator with a mechanical coupling to the base, an external pump forming part of a fluid circulation system, or any other device for causing fluid circulation within the bioreactor 100. Regardless of form, the resulting agitation causes fluid to flow throughout the bioreactor 100, such as upwardly into a central chamber 126 forming a cell culturing zone, in this version, which may include a fixed bed for culturing cells.

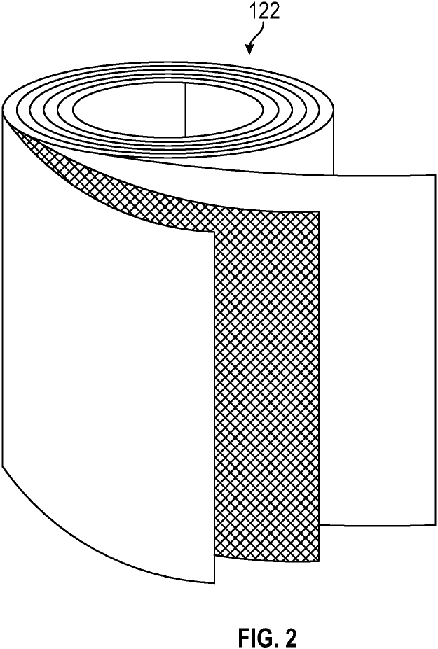

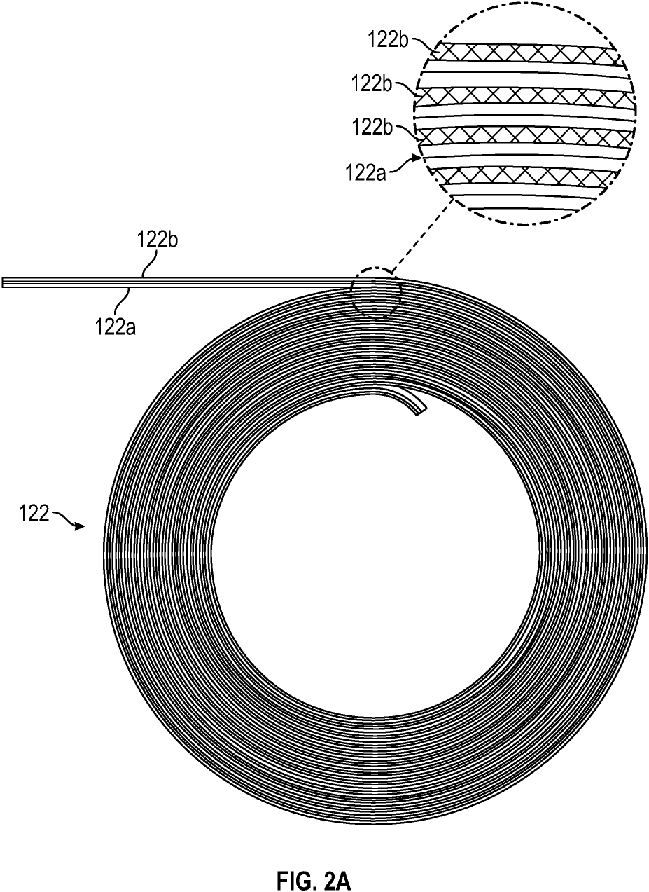

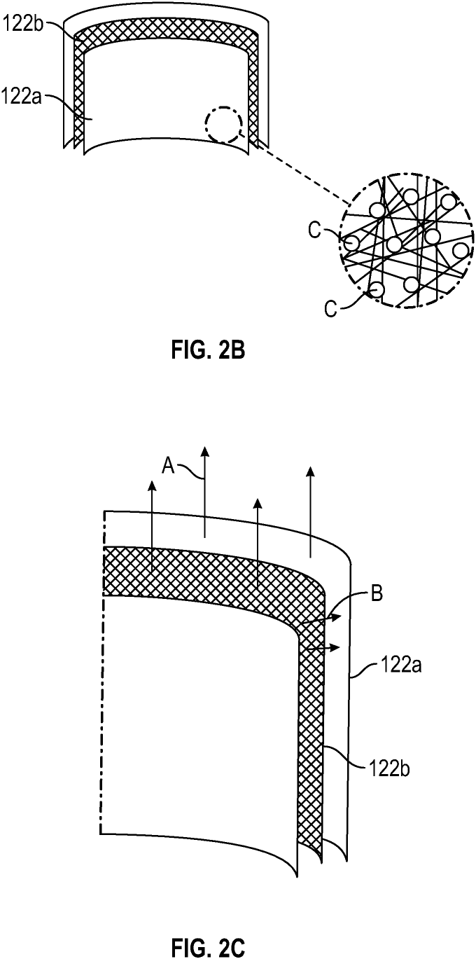

FIG. 2 shows a fixed bed in the form of a structured spiral bed 122 which, in use, may contain and retain cells being grown. In some embodiments, the spiral bed 122 may be in the form of a cartridge that may be built within and as a part of or introduced into the outer chamber 120 (but as noted further below may also be located in the central chamber in alternative embodiments). The bed 122 can be pre-installed in the bioreactor 100 during manufacture at a facility prior to shipping or installed at the point of use.

FIG. 2A shows one embodiment of a matrix material for use as a structured fixed bed in the bioreactor of the present disclosure and, in particular, a spiral bed 122. One or more cell immobilization layers 122a (woven or non-woven) are provided adjacent to one or more spacer layers 122b made from a mesh structure. The layering may optionally be repeated several times to achieve a stacked or layered configuration, and the spacer layers 122b are considered optional.

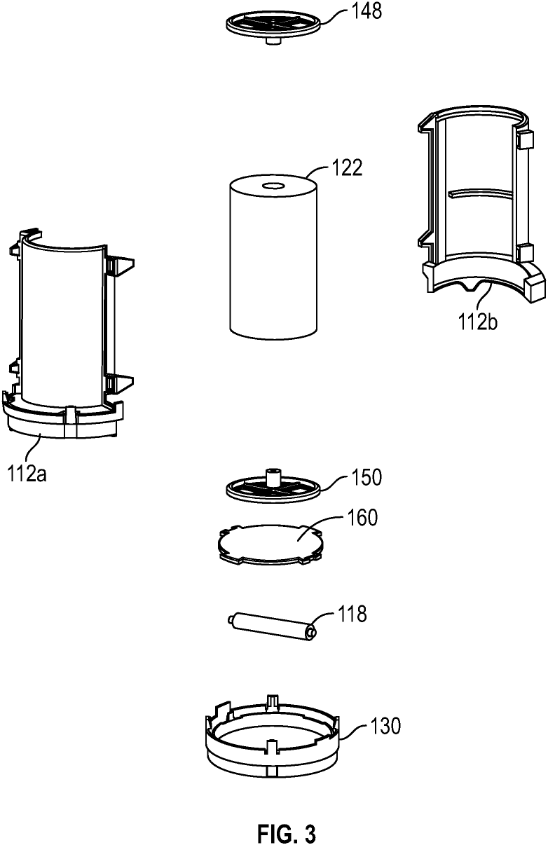

The mesh structure included in spacer layers 122b forms tortuous paths to steer the cells into the depth of the cell immobilization layers 122a (see cells C in FIG. 2A suspended or entrapped in the material of the immobilization layer 122a). As shown in FIGS. 2B and 2C, the spacer layers 122b also form channels 122c in conjunction with the adjacent cell immobilization layers 122a for fluid and bubbles to flow therethrough (see arrows A in FIG. 3B indicative of flow between layers, and also arrows B in FIG. 2C indicative of transverse flow). Increased homogeneity of the cells is maintained within the structured fixed bed as a result of this type of arrangement. Other spacer structures can be used which form such tortuous paths, and again, the spacer layer is considered entirely optional (and, if omitted, the spiral fixed bed 122 may comprise a single cell immobilization layer 122b, and especially one formed of a woven material).

The layers 122a, 122b (one or both) can be subsequently spirally or concentrically rolled along an axis or core (e.g., conduit or tube 128, which may be provided one or more component parts). The layers 122a, 122b of the structured fixed bed 122 may be firmly wound. In some embodiments, the diameter of the core (tube 128), the length and/or amount of the layers will ultimately define the size of the assembly or matrix. In some embodiments, thickness of each of the layers 122a, 122b may be between 0.1 and 5 mm, 0.1 and 10 mm, or 0.001 and 15 mm.

The orientation of the fixed bed 122 may be other than as shown in a bioreactor 100 as shown in FIG. 2, where the flow is arranged vertically (bottom to top, in the example provided). For example, the structured fixed bed 122 may be comprised of one or more horizontally arranged material layers. The one or more layers may comprise a woven or reticulated material. The fixed bed 122 may also comprise a three-dimensional (3D) monolith matrix, such as if the form of a scaffold or lattice formed of multiple interconnected units or objects (e.g., round or spherical beads connected by connectors), which objects have surfaces for cell adhesion. The fixed bad may also comprise an unstructured fixed bed.

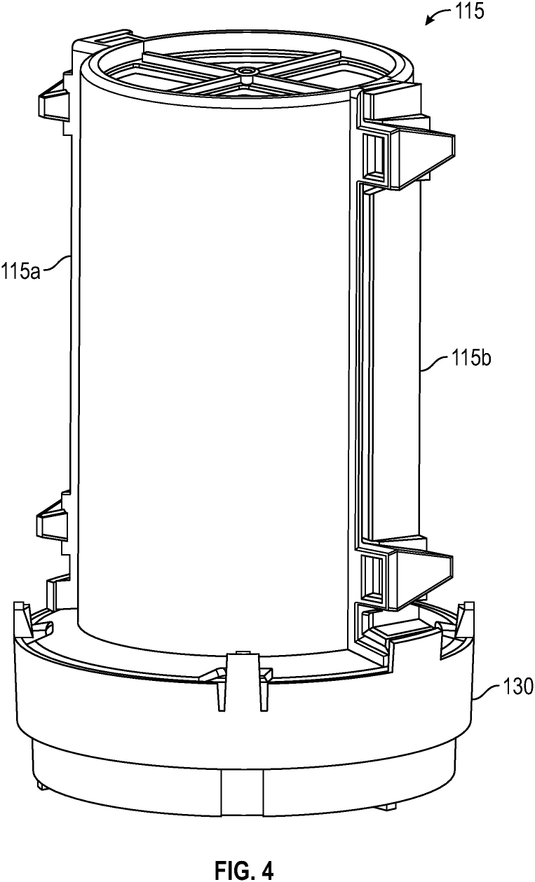

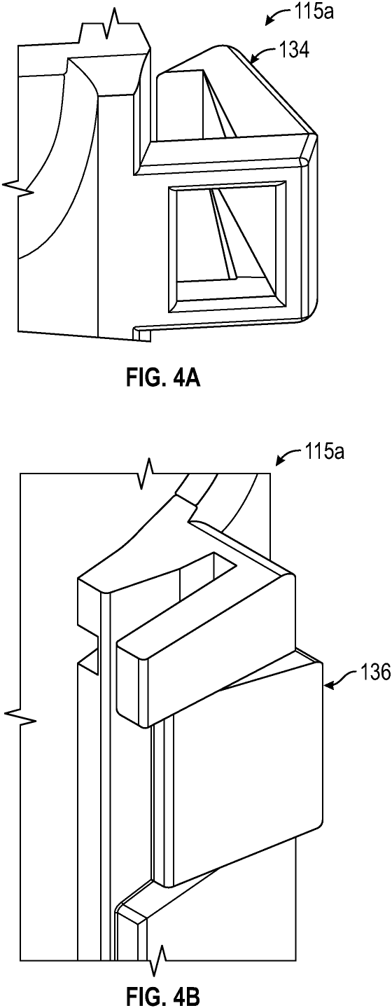

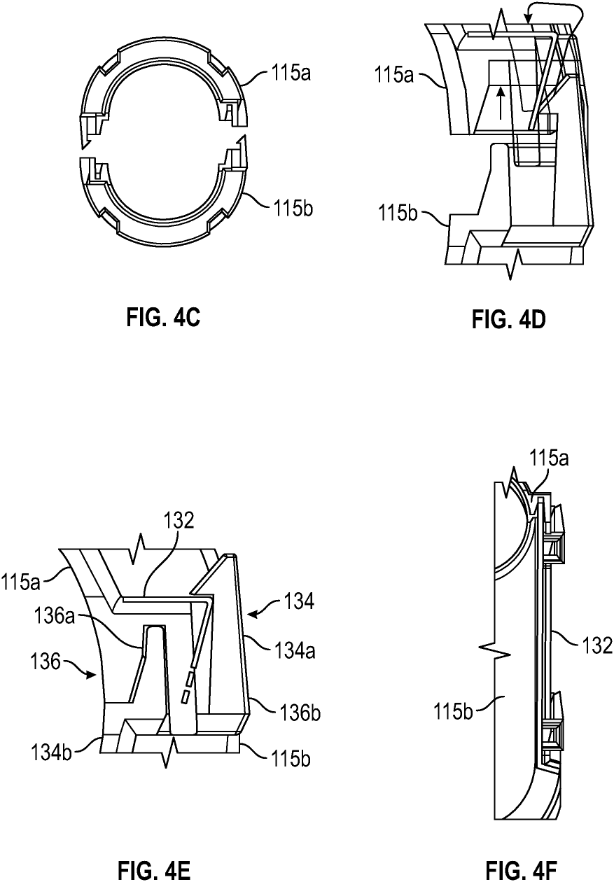

According to one aspect of the disclosure, and with reference to FIG. 3, the container 115 forming the cell culture vessel may be a modular component. Specifically, to allow for a degree of dimensional flexibility in the assembly, the container 115 may be made from a plurality of interconnectable portions, such as for example first and second semi-circular portions 115a, 115b, which may comprise halves of the container (but which may not comprise exactly one half of the container in terms of area). The portions 115a, 115b may mate together and interconnect to form a tubular body for connecting with a base 130, as shown in FIGS. 3 and 4 (but not limited to a particular cross-section for the tubular body, which is shown as circular merely by example, and could take other shapes, without limitation).

In order to interconnect these portions 115a, 115b, while preventing or significantly reducing the amount of leakage at the resulting connection, a sealing arrangement providing a tortuous path or labyrinth may be used. This may involve providing each portion 115a, 115b with a mating male coupler 134 and female coupler 136. As shown in FIGS. 4C, 4D, and 4E, the male coupler 134 on one side of portion 115a includes an active or locking male prong 134a for engaging a ledge 136b, and the other portion 115a at a corresponding side includes a passive male prong 134b for fitting into a female recess 136a. The alternate sides include a similar, but reversed arrangement, and which pattern may repeat along the height of the container 115 as well (four such instances shown, with each mating pair at a different height). Together, these cooperating structures form a releasable connection and a corresponding sealing arrangement having a tortuous passage 132 that prevents fluid from readily passing.

As can be understood, this arrangement allows the container 115 to be manufactured in a modular fashion for later ease of assembly (or disassembly) and insertion into an outer vessel to form the bioreactor 100, without allowing for any appreciable (or at least controlled) leakage (note arrow L) at the resulting seams. This may be achieved without the need for adhesives, separate gaskets/O-rings, or other forms of attachment (such as welding or mechanical fasteners). From FIG. 4F, it can be understood that the sealing arrangement may extend the entire height of the container, if desired, but could also extend along only a portion of it, if desired for a particular application.

The formation of the container 115 in this manner also may provide further advantages. For instance, no draft angle exists inside the housing 112 and thus no risk of having by-pass flow between the fixed bed 122 and the internal wall of the housing 112. Moreover, it is very easy to insert a fixed bed 122, made of a spiral, inside the half part than inside a straight cylinder or a cone. Specifically, it is possible to simply drop the fixed bed 122 inside the first portion 115a and it by the second portion 115b, which avoids the risk of not having a good spiral at the end or the risk of damage the materials during the insertion.

While two portions 115a, 115b are shown, it can be appreciated that more than two portions may be used to form the container 115, with each point of contact, or seam, including such a sealing arrangement. The arrangement is shown as being applied to vertically oriented portions 115a, 115b of the container 115, but it may be applied to any other structures in the bioreactor 100, without limitation, including for example the external casing or housing 112 forming the outer vessel.

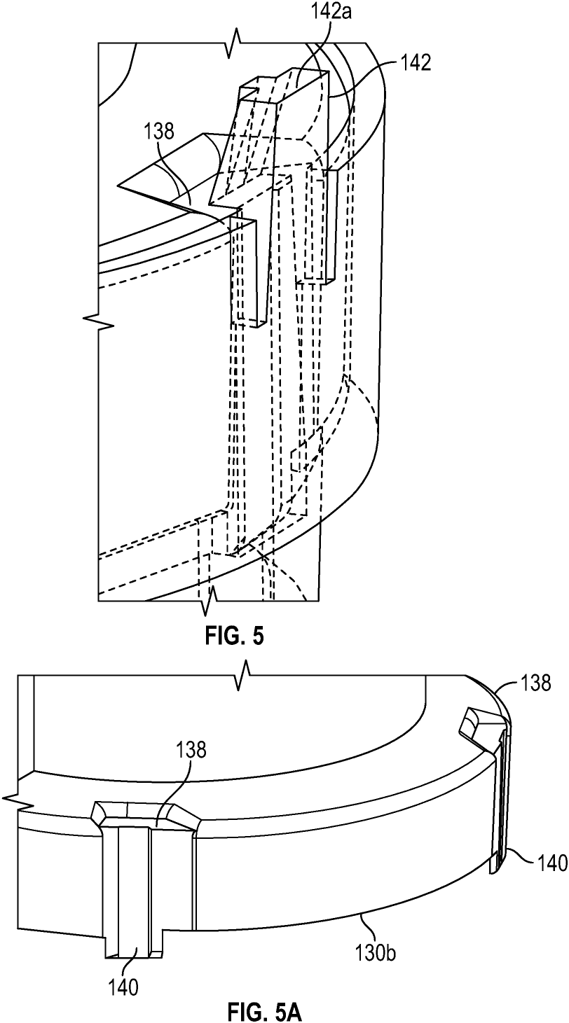

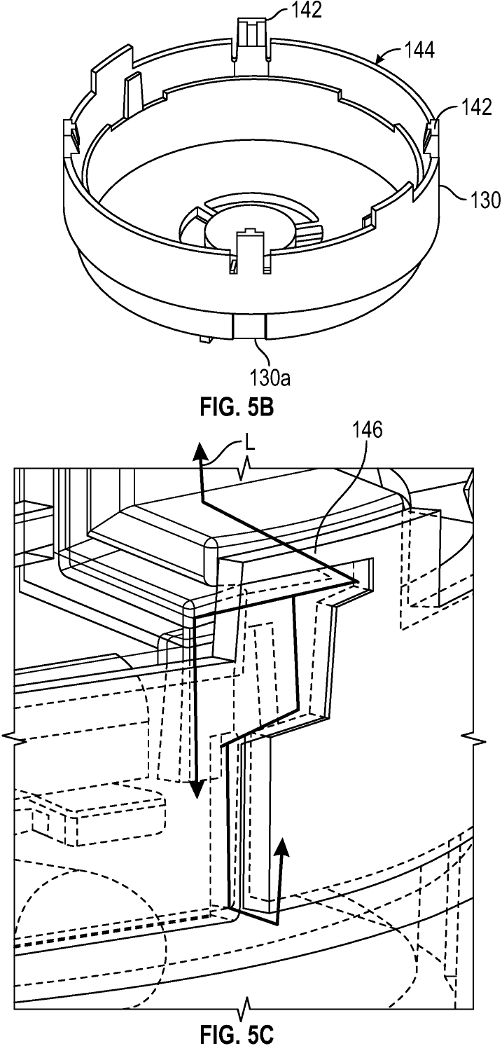

The portions 115a, 115b may also be adapted, when coupled, to releasably connect with a third portion, such as the base 130. With reference to FIGS. 5, 5A, 5B, and 5C, the portions 115a, 115b may include recesses 138 associated with depending projections 140. The base 130 may include corresponding flexible fingers 142, each having an oversized head portion 142a that may create a snap-fit engagement with the recesses 138, while depending projections 140 extend into adjacent openings 130a formed in the base and ensure proper alignment and seating.

From FIG. 5B, it can be also understood that the base 130 includes an inner recess 144 for receiving a depending portion 130b of the base in a seated engagement when the fingers 142 are locked in position. From FIG. 5C, it can be understood that this mode of connection likewise creates a labyrinth sealing arrangement, with a tortuous path 146 thus formed between the respective parts. Despite the lack of seals or adhesives, this helps to control any liquid leakage at the connection points.

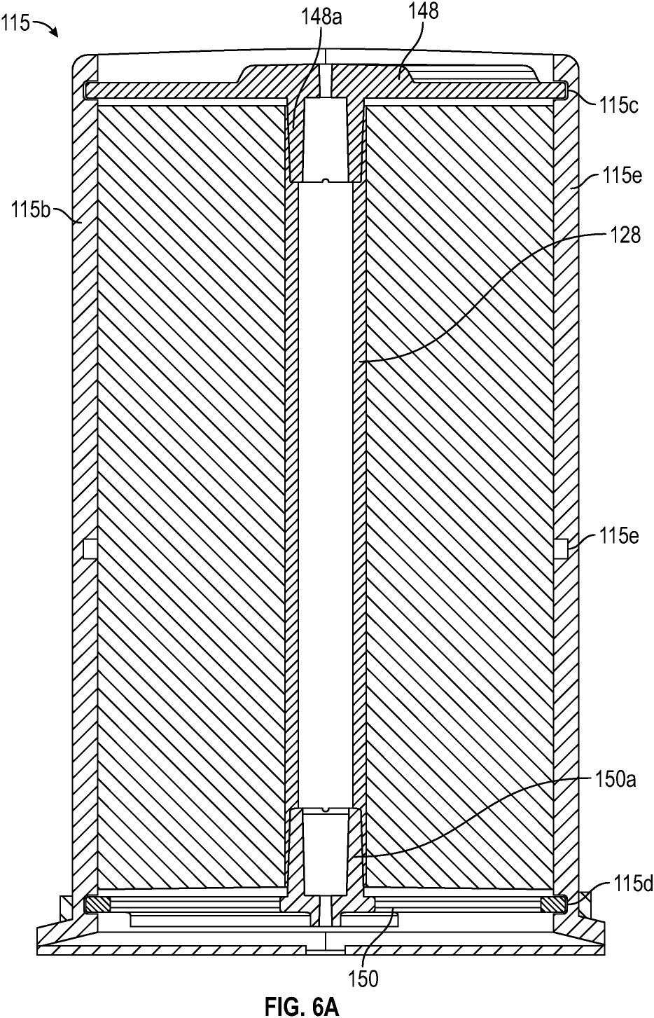

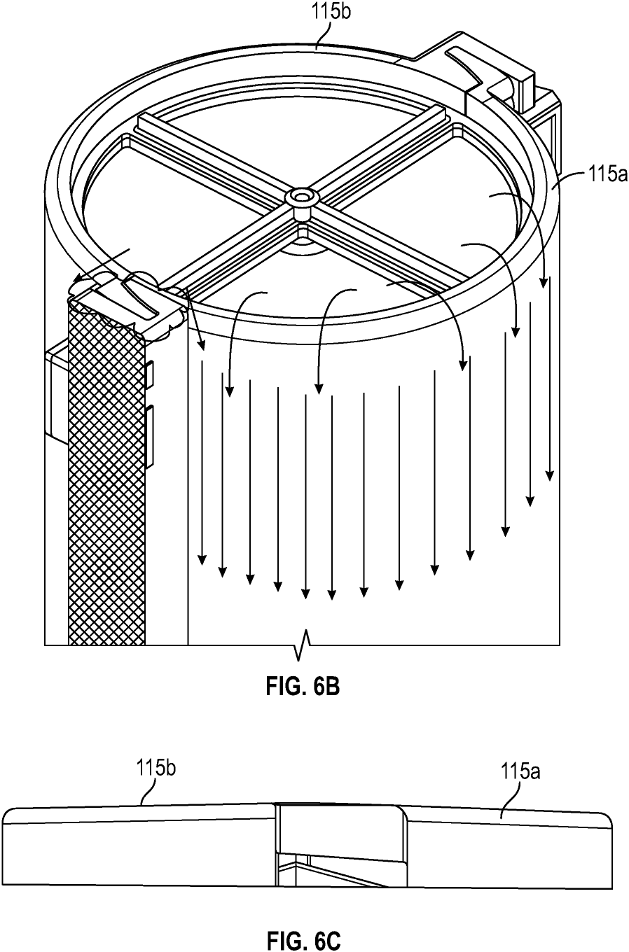

Turning now to FIGS. 6A, 6B, and 6C, the manner in which a fixed bed (not shown) may be retained in the container 115 is shown. Specifically, the portions 115a, 115b of the container 115 may be provided with internal receivers in the form of upper and lower grooves 115c, 115d. These grooves 115c, 115d may receive and engage upper and lower retainers 148, 150, which may be secured to the central tube 128 about which the fixed bed (not shown) is formed (such as by spirally wrapping of one or more layers 122a, 122b, as shown in FIG. 2). The securement may be achieved by way of projecting connectors in the nature of hose barbs 148a, 150a that are removably received in the tube 128, such as by way of press-fit connection. The portions 115a, 115b may thus be interconnected over the retainers 148, 150 and the central tube 128 during the assembly of the container 115, prior to insertion into a bioreactor.

One or more additional receivers, such as a central groove 115e, may also be provided for engaging one or more additional retainers, such as if there exists a desire to provide multiple fixed beds in a stacked configuration (as outlined further in the description that follows). It can also be appreciated from FIGS. 6A, 6B, and 6C that the upper edges of the portions 115a, 115b may be drafted during the molding process to help liquid flow over the edge in in an efficient manner, and to discourage liquid from accumulating on the mating connectors.

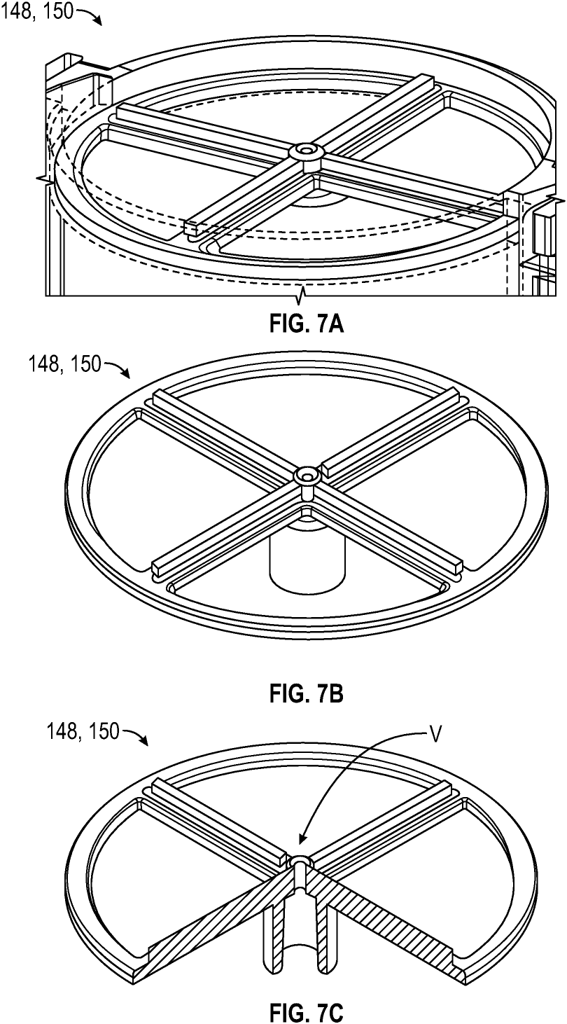

FIGS. 7A, 7B, and 7C further illustrate how the retainers 148, 150 may be formed. Each retainer 148, 150 may include a peripheral portion that matches the inner shape and diameter of the container 115, so as to fit snugly within the corresponding receiver (e.g., groove 115c or 115d). The retainers 148, 150 are also adapted for holding the fixed bed (not shown) in position, while allowing fluid to pass, which may be achieved by the cross-members illustrated, but a perforated plate could also be used for this purpose. As perhaps best understood from FIGS. 6 and 7C, a central vent V may also be provided to allow for air or liquid to escape the central tube 128.

Turning back to FIGS. 1 and 3, it can also be understood that a retainer may be associated with the base 130 for retaining the agitator, such as stir bar 118, therein. In the illustrated version, the retainer comprises a plate 160 with peripheral notches for allowing fluid to pass when mounted to the base 130, such as by outwardly directed tabs 160a for engaging corresponding receivers in the base. In this manner, the agitator (e.g., stir bar 118) is retained securely in place, but the arrangement is subject to simple disassembly, if desired for cleaning or other servicing, as examples. It can also be understood from FIG. 1 that the base 130 includes fluid inlets I for allowing fluid to enter the chamber 116 from a space between a floor of the housing 112 and the underside of the base, and thus reach the fixed bed of container 115 via the peripheral notches when connected thereto.

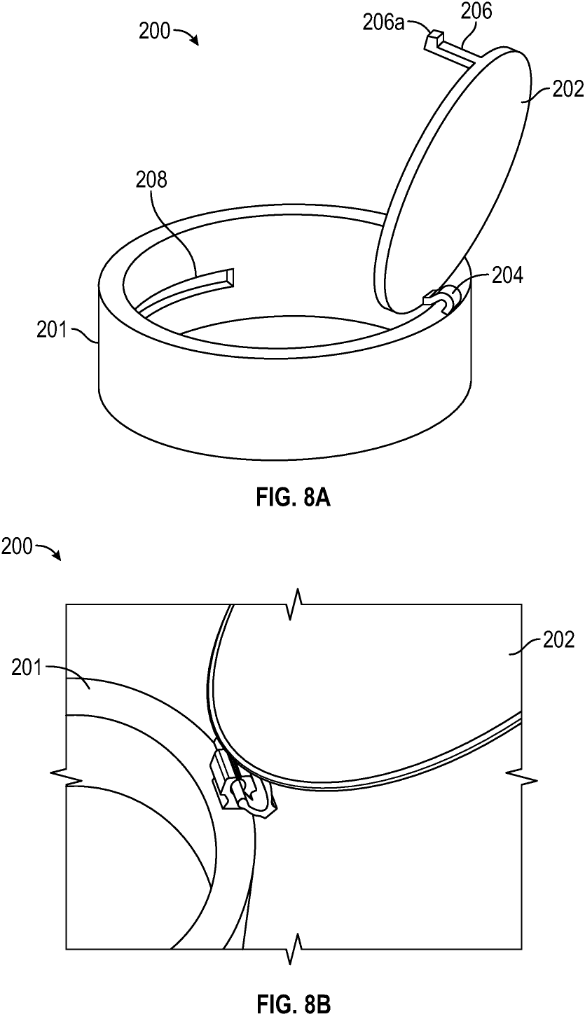

According to a further, and possibly related aspect of the disclosure, and with reference to FIGS. 8A and 8B, a removable cap 200 may be provided for connecting to the upper portion of the housing 112, such as by way of friction fit or similar removable engagement. The cap 200 may include a tubular body 201 connected to a covering in the form of a perforated plate 202, which is removably connected to allow for insertion of the fixed bed 122 into the corresponding chamber 120 of the bioreactor 100. The connection may be by way of a hinge 204 (which may be a one-piece, living type of hinge, as shown in FIG. 8A, or a two-piece, separable arrangement, as shown in FIG. 8B). A small groove (or portion of groove) can be provided on the external wall of one or both portions 115a, 115b to help retain the cap 200 in place.

A releasable locking arrangement may also be provided for retaining the perforated plate 202 in the deployed condition. For example, this plate 202 may be provided with a depending leg 206. The leg 206 may include a latch 206a for engaging a corresponding groove 208 in the cap 200.

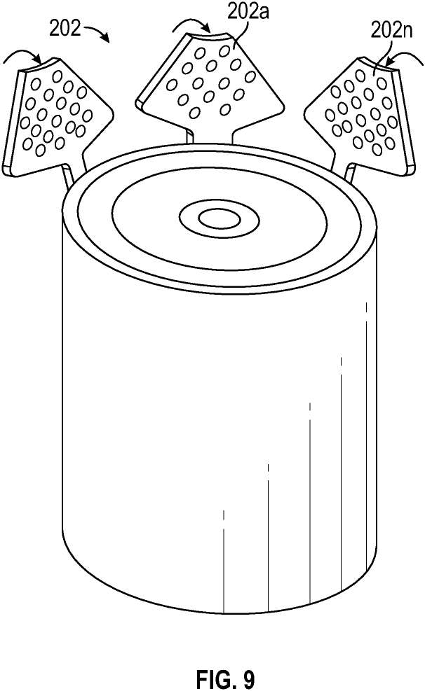

An alternative version in which the perforated plate 202 is made from a plurality of portions 202a ... 202n, each of which may be hingedly connected to the cap 200, is shown in FIG. 9. These portions 202a ...202n may mate in the deployed position covering the cap 200 to form the plate 202, and may be individually raised and lowered to provide access to the underlying chamber or compartment (which may include the fixed bed, and thus selectively raising and lowering could be used for sampling purposes).

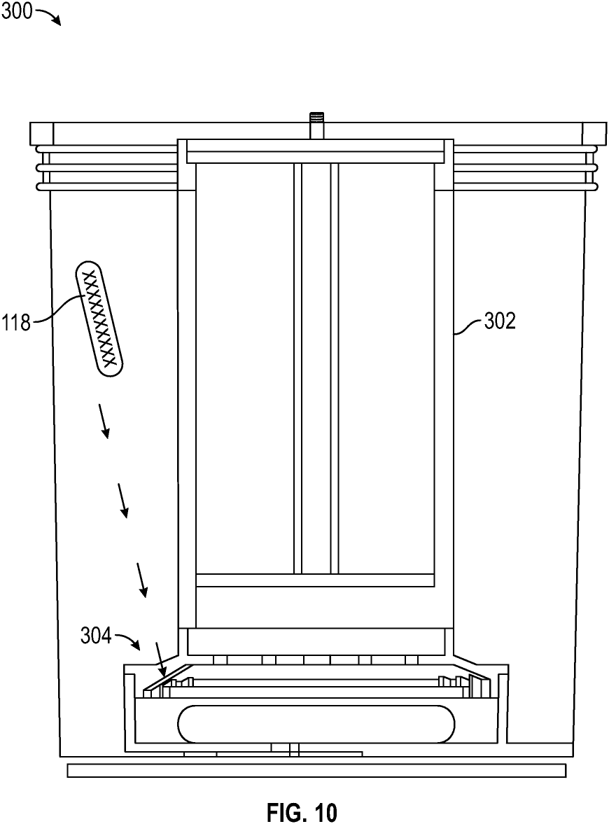

According to a further aspect of the disclosure, and with reference to FIGS. 10-12, an embodiment of a modular bioreactor 300 is shown. In this embodiment, an internal container 302 includes a door 304. This door 304 may be used to introduce a component, such as an agitator (stir bar 118), into the associated chamber once assembled, and without the need to remove the container 302 from the bioreactor 300.

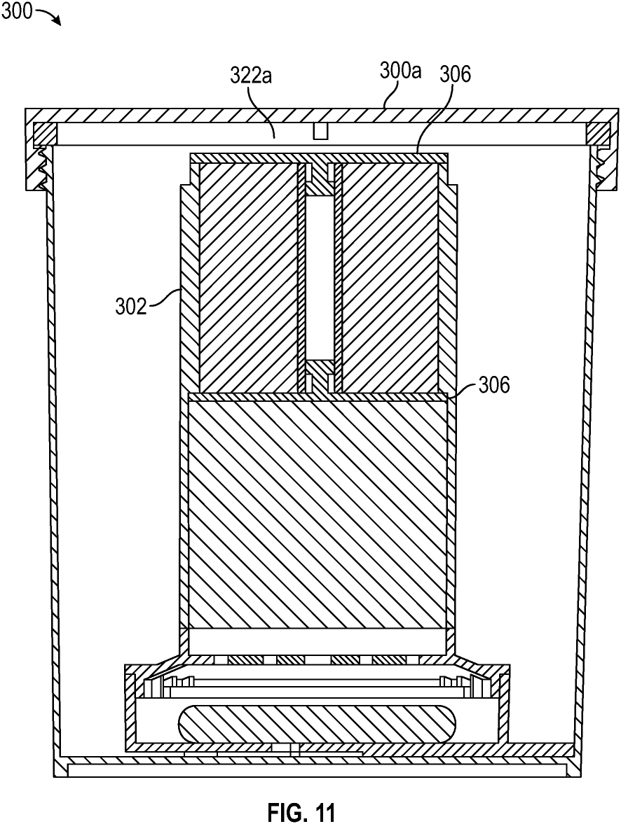

A further embodiment of the internal container 302 for a bioreactor 300 is shown in FIG. 11. Instead of using grooves, dividers or plates 306 in this version are each provided with a connector, such as for example a barb 300a, adapted for releasably engaging the central core 322a. This allows for the plates 306 to have a degree of axial freedom, and also allows for the plates 306 to be identical in construction and thus interchangeable. The bed 322 itself may also have a central core 322a, which may be hollow and allow for receiving a structure, such as a probe, for taking measurements.

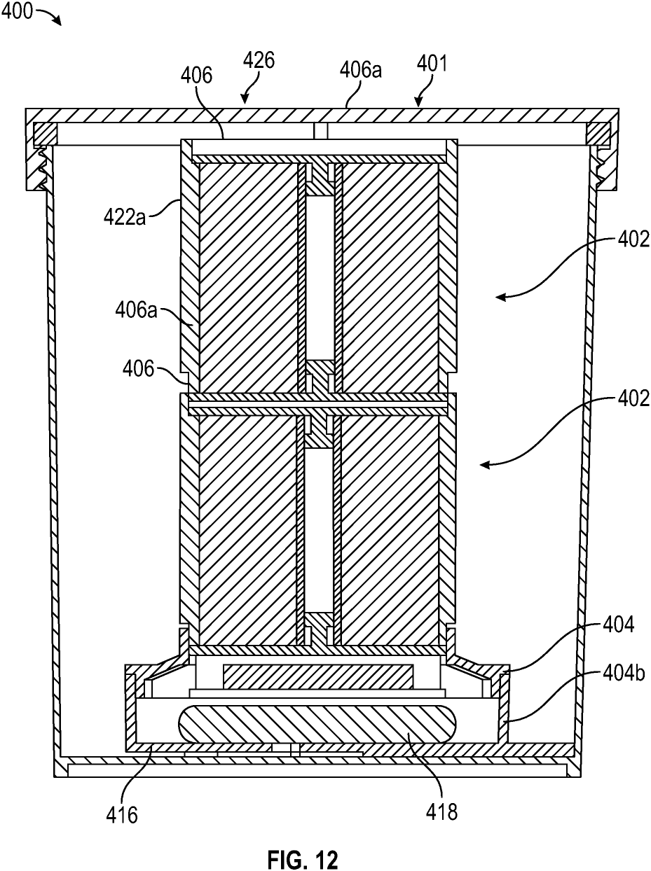

Turning now to FIGS. 12-14, a further aspect of the disclosure is illustrated. These figures illustrate a bioreactor 400 in which a removable divider 401 comprises one or more cartridges 402 forming a central column 426. One or more of the cartridges 402 may include a fixed bed 422, such as the spiral version previously described. The cartridge(s) 402 may be constructed as shown in FIG. 11, with upper and lower perforated plates 406 each including connectors (such as hose barb 406a) for insertion into a central core 422a. The cartridge(s) 402 may be adapted for nesting together in a stacked arrangement, such that liquid may freely pass from one to the other via plates 406.

The cartridges 402 may be fixed inside the bioreactor 400 in a variety of ways. For example, the lower cartridge 402 (or possibly the only one) may be adapted to connect to (nest with) the housing 404 for receiving the agitator, such as stir bar 418. This housing 404 may be adapted to receive liquid from the outer chamber 420 and direct it to the lower entrance end of the lower cartridge 402.

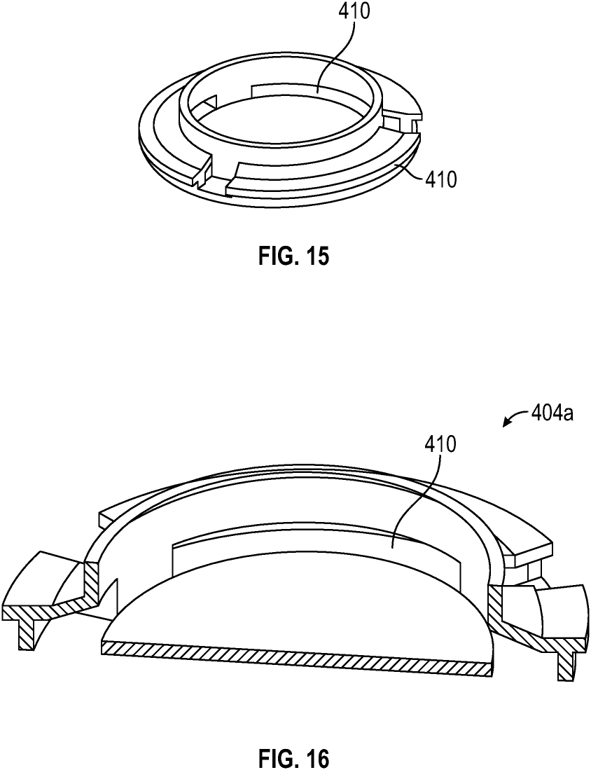

The cartridges 402 may be of similar heights. While two cartridges are shown in a vertically stacked, tandem configuration, any number may be provided depending on the desired arrangement of the bioreactor 400. Each cartridge 402 may be formed of separable portions or halves, which may be releasably locked together (see, e.g., FIG. 15).

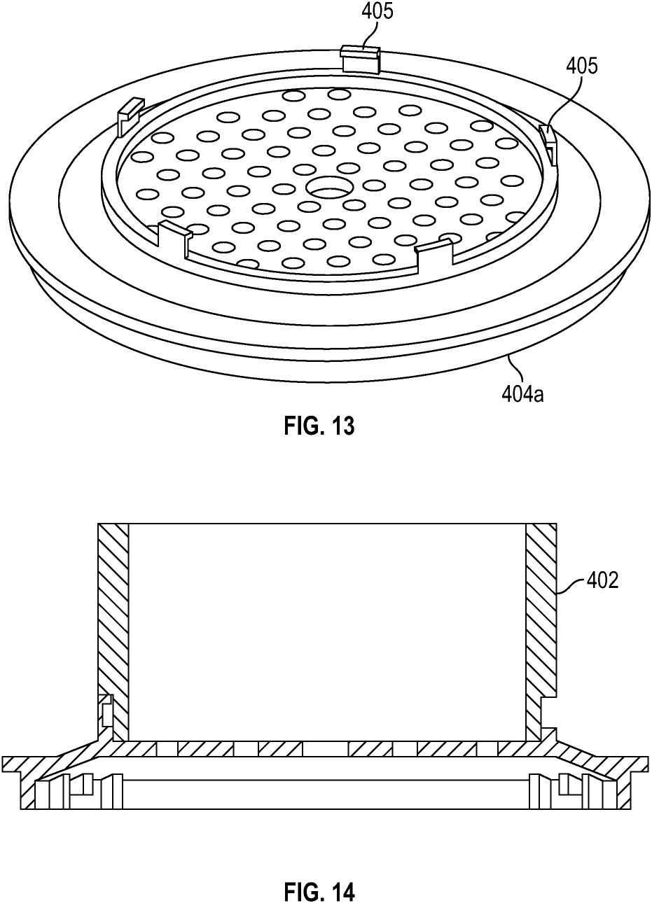

FIGS. 13 and 14 shows that the retainer forming an upper part of the housing 404 shown in FIG. 12 may comprise a single portion 404a adapted to connect to a lower portion 404b forming a chamber 416 for the agitator, such as a stir bar 418. This single portion 404a may also include clips 405 for connecting with the cartridge 402. The portion 404a may be perforated, as shown in FIG. 13, or as shown in FIGS. 15 and 16, may include peripheral openings 410 for allowing agitated liquid to flow vertically into the cartridge(s) 402 as disclosed, or any other form of fixed bed.

Summarizing, this disclosure may be considered to related to the following items:

1. An apparatus for culturing cells, comprising:
a housing having an inner compartment; and
a container for removable positioning in the inner compartment of the housing, the container comprising first and second portions adapted to interlock for containing a fixed bed for culturing cells, the container forming a chamber within the inner compartment for circulating fluid to or from the fixed bed.

2. The apparatus of item 1, wherein the first and second portions comprise interlocking male and female couplers.

3. The apparatus of item 1 or item 2, wherein the first and second portions include a sealing arrangement forming a tortuous path for fluid flow.

4. The apparatus of any of items 1-3, wherein the removable container includes one or more internal grooves for engaging retainers for retaining the fixed bed therein.

5. The apparatus of item 4, wherein the container comprises a central tube, the retainers adapted for engaging the central tube.

6. The apparatus of item 5, wherein each of the retainers includes a barb adapted for insertion into an open end of the central tube.

7. The apparatus of any of items 1-6, wherein the removable container further includes a third portion for receiving an agitator.

8. The apparatus of item 7, wherein the third portion includes a door for receiving the agitator.

9. The apparatus of item 7 or item 8, wherein the third portion including one or more openings for allowing fluid flow to enter the container.

10. The apparatus of any of items 7-9, wherein the third portion is adapted to relasably connect to the container.

11. The apparatus of any of items 7-10, further including a retainer comprising a plate with peripheral notches for allowing fluid to pass to the third portion of the container.

12. The apparatus of any of items 1-11, further including a cap for engaging the container, the cap comprising a hinged plate.

13. The apparatus of item 12, wherein the hinged plate comprises a releasable locking arrangement for locking with the cap in a closed position.

14. The apparatus of any of items 1-13, wherein the first and second portions comprise halves of the container.

15. An apparatus for culturing cells, comprising:

a fixed bed comprising a retainer at one end and a tube passing centrally through the fixed bed, the tube including an open end for receiving a portion of the retainer.

16. The apparatus of item 15, wherein the fixed bed comprises a spiral fixed bed.

17. The apparatus of item 15 or item 16, further including a tubular body for receiving the fixed bed, the tubular body having an internal groove for receiving the retainer.

18. The apparatus of item 17, further including a cap for engaging the tubular body, the cap comprising a hinged plate.

19. The apparatus of item 18, wherein the hinged plate comprises a releasable locking arrangement for locking with the cap in a closed position.

20. A method of assembling a bioreactor, comprising:
interconnecting first and second portions to form a container including a fixed bed; and
inserting the container into an inner compartment of a housing to form a chamber therein circulating fluid to or from the fixed bed.

21. The method of item 20, further including the step of providing a retainer on opposed ends of the fixed bed prior to the inserting step.

22. The method of item 21, further including the step of connecting the retainers to the first and second portions of the container prior to the inserting step.

23. The method of item 21 or item 22, further including the step of inserting a portion of the retainers into a tube positioned within the fixed bed.

24. The method of any of items 20-23, further including the step of connecting a base portion including an agitator to the container prior to the inserting step.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/- 20% or less, preferably +/-10% or less, more preferably +/-5% or less, even more preferably +/-1% or less, and still more preferably +/-0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

While preferred embodiments according to the disclosed concepts have been shown and described herein, it will be obvious to those skilled in the art that such embodiments are provided by way of example only. Numerous variations, changes, and substitutions will now occur to those skilled in the art without departing from the invention. For example, while the bioreactor is shown in a vertical orientation, it could be used in any orientation. It should be understood that various alternatives to the embodiments of the invention described herein may be employed in practicing the invention. It is intended that the following claims define the scope of the protection under the applicable law and that methods and structures within the scope of these claims and their equivalents be covered thereby.

What is claimed:

1. An apparatus for culturing cells, comprising:
a housing having an inner compartment; and
a container for removable positioning in the inner compartment of the housing, the container comprising first and second portions adapted to interlock for containing a fixed bed for culturing cells, the container forming a chamber within the inner compartment for circulating fluid to or from the fixed bed.

2. The apparatus of claim 1, wherein the first and second portions comprise interlocking male and female couplers.

3. The apparatus of claim 1, wherein the first and second portions include a sealing arrangement forming a tortuous path for fluid flow.

4. The apparatus of claim 1, wherein the removable container includes one or more internal grooves for engaging retainers for retaining the fixed bed therein.

5. The apparatus of claim 4, wherein the container comprises a central tube, the retainers adapted for engaging the central tube.

6. The apparatus of claim 5, wherein each of the retainers includes a barb adapted for insertion into an open end of the central tube.

7. The apparatus of claim 1, wherein the removable container further includes a third portion for receiving an agitator.

8. The apparatus of claim 7, wherein the third portion includes a door for receiving the agitator.

9. The apparatus of claim 7, wherein the third portion including one or more openings for allowing fluid flow to enter the container.

10. The apparatus of claim 7, wherein the third portion is adapted to releasably connect to the container.

11. The apparatus of claim 7, further including a retainer comprising a plate with peripheral notches for allowing fluid to pass to the third portion of the container.

12. The apparatus of claim 1, further including a cap for engaging the container, the cap comprising a hinged plate.

13. The apparatus of claim 1, wherein the hinged plate comprises a releasable locking arrangement for locking with the cap in a closed position.

14. The apparatus of claim 1, wherein the first and second portions comprise halves of the container.

15. An apparatus for culturing cells, comprising:
a fixed bed comprising a retainer at one end and a tube passing centrally through the fixed bed, the tube including an open end for receiving a portion of the retainer.

16. The apparatus of claim 15, wherein the fixed bed comprises a spiral fixed bed.

17. The apparatus of claim 15, further including a tubular body for receiving the fixed bed, the tubular body having an internal groove for receiving the retainer.

18. The apparatus of claim 15, further including a cap for engaging the tubular body, the cap comprising a hinged plate.

19. The apparatus of claim 18, wherein the hinged plate comprises a releasable locking arrangement for locking with the cap in a closed position.

20. A method of assembling a bioreactor, comprising:
interconnecting first and second portions to form a container including a fixed bed; and
inserting the container into an inner compartment of a housing to form a chamber therein circulating fluid to or from the fixed bed.

21. The method of claim 20, further including the step of providing a retainer on opposed ends of the fixed bed prior to the inserting step.

22. The method of claim 21, further including the step of connecting the retainers to the first and second portions of the container prior to the inserting step.

23. The method of claim 21, further including the step of inserting a portion of the retainers into a tube positioned within the fixed bed.

24. The method of claim 20, further including the step of connecting a base portion including an agitator to the container prior to the inserting step.
